(19) United States
(12) Patent Application Publication
(10) Pub. No.: US 2002/0164637 A1
(43) Pub. Date: Nov. 7, 2002

(54) SCREEN FOR ECDYSONE RECEPTOR LIGANDS

(76) Inventors: PAUL MAK, POMONA, NY (US); FERNANDO E. DELA CRUZ, FAIRLESS HILLS, PA (US)

Correspondence Address: SUTHERLAND ASBILL & BRENNAN LLP, 999 PEACHTREE STREET, N.E., ATLANTA, GA, 30309, US

(21) Appl. No.: 08/865,961

(22) Filed: May 30, 1997

Related U.S. Application Data
(60) Provisional application No. 60/018,631, filed on May 31, 1996.

(51) Int. Cl. G01N 33/53

(52) U.S. Cl. 435/7.1

(57) ABSTRACT

The present invention encompasses a method for identifying ecdysone receptor (EcR) ligands. The method is carried out by:

(i) providing an extract derived from a transformed yeast cell, wherein the extract comprises

(a) EcR or a functional derivative thereof; and

(b) an EcR binding partner or a functional derivative thereof;

(ii) measuring specific binding to the extract of an authentic EcR ligand, in the absence and presence of a test compound; and

(iii) identifying as an EcR ligand a test compound that reduces the specific binding of the authentic EcR ligand to the extract.

### FIELD OF THE INVENTION

The present invention relates to the identification of compounds that bind to the ecdysone receptor (EcR), which are useful as insecticides or as lead compounds for the development of insecticides. The invention provides methods and compositions for the identification of such compounds.

### BACKGROUND OF THE INVENTION

In Drosophila, complete metamorphosis requires a pulse of the steroid molting hormone, 20-hydroxyecdysone, at the end of the larval stage (Richards, Biol.Rev. 56:501, 1981). The molecular mechanism of ecdysone action has been studied extensively since the early observations of its effects on polytene chromosome puffing (Ashburner et al., Cold Spring Harbor Symp. Quant. Biol. 38:655, 1974). The cloning of the Drosophila ecdysone receptor (EcR) gene revealed that it is a member of the steroid/thyroid/retinoid receptor superfamily of ligand-activated transcription factors (Koelle et al., Cell 67:59, 1991). However, unlike other classical steroid hormone receptors such as estrogen, androgen or progesterone receptors, DNA-binding and target gene activation of the EcR requires the presence of its heterodimeric partner, ultraspiracle protein (Usp) or CF1, the insect homologue of vertebrate retinoid X receptors and specific ligands (Ora et al., Nature 347:298, 1990; Shea et al., Genes Dev. 4:1128, 1990; Yao et al., Nature 366:476, 1993). Although this observation indicates that Usp is an essential component of the EcR function, the precise signaling event prior to target gene activation by these heterodimeric complexes is still unknown.

The present inventors have found that ligand binding to EcR expressed in a heterologous cell requires co-expression in the same cell of Usp. Prior to this finding, it was not possible to use heterologous EcR expression systems to identify EcR ligands. The present invention provides a means of screening a multiplicity of test compounds to identify those that bind EcR-Usp complexes.

The critical role of EcR and EcR-Usp complexes in insect development implicates EcR as a target for environmentally safe compounds that act as insect growth regulators (IGRs) (Graf, Parasitology Today 9:471, 1993). Ecdysone itself has been proposed for such a use, but is too complex and costly to manufacture. In addition, insects possess enzyme systems that catabolize ecdysones (Kerkut et al., eds., Comprehensive Insect Physiology, Biochemistry, and Pharmacology: Endocrinology I Vol.7, 363, 1985). Accordingly, it would be advantageous to identify compounds that are less expensive to produce and are not cleared by insects. One such compound, RH5849 (Rohm and Haas) is a non-steroidal compound with ecdysone-like activity; however, this compound has limited application as an insecticide, because it is at least 100-fold less active in bioassays, and at least 30-fold less active in binding assays, compared with 20-hydroxyecdysone (Wing, Science 241:467, 1988).

Thus, there is a need in the art for compositions and methods, including high-throughput methods, useful for identifying compounds that bind EcR and mimic the action of ecdysone.

### SUMMARY OF THE INVENTION

The present invention encompasses a method for identifying ecdysone receptor (EcR) ligands. The method is carried out by:

(i) providing an extract derived from a transformed yeast cell, wherein the extract comprises

(a) EcR or a functional derivative thereof; and

(b) an EcR binding partner or a functional derivative thereof;

(ii) measuring specific binding to the extract of an authentic EcR ligand, in the absence and presence of a test compound; and

(iii) identifying as an EcR ligand a test compound that reduces the specific binding of the authentic EcR ligand to the extract.

Authentic EcR ligands are compounds that bind with high affinity and specificity to EcR and/or transcriptionally activate ecdysone-regulated genes. Test compounds are compounds being evaluated for their ability to bind EcR and mimic ecdysone action. Preferably, the EcR binding partner is Usp. Derivatives of both EcR and Usp may be used, so long as they are able to form a functional complex that retains its capacity to bind authentic EcR ligands.

In one embodiment of the invention, the extract containing EcR and an EcR binding partner is contacted with a radiolabelled authentic EcR ligand, and unlabelled test compounds are used to reduce the amount of radioactivity specifically bound to the yeast extract. Preferably, the method of the invention is employed in a high-throughput mode, allowing the screening of a multiplicity of test compounds in a single assay.

### BRIEF DESCRIPTION OF THE DRAWINGS

FIG. 1A is a photographic illustration of immunoblot analysis of extracts derived from S. cerevisiae expressing Drosophila melanogaster ecdysone receptor (EcR) and ultraspiracle protein (Usp). Yeast extracts (50 μg protein) prepared from yeast strains expressing EcR (yE), Usp (yU) or both (yE/yU) were resolved by 10% SDS-PAGE. Proteins were transferred onto nitrocellulose membranes, which were probed with a monoclonal antibody (AB11) against Usp. The arrows indicate the molecular size of yeast expressed Usp.

FIG. 1B is a photographic illustration of immunoblot analysis of extracts derived from S. cerevisiae expressing Drosophila melanogaster ecdysone receptor (EcR) and ultraspiracle protein (Usp). Yeast extracts (50 μg protein) prepared from yeast strains expressing EcR (yE), Usp (yU) or both (yE/yU) were resolved by 10% SDS-PAGE. Proteins were transferred onto nitrocellulose membranes, which were probed with a monoclonal antibody (AD4.4) against the EcR. The arrows indicate the molecular size of yeast expressed EcR.

FIG. 2 is a graphic representation of the specific binding of ³H-Ponasterone A to extracts derived from yeast strains expressing EcR alone; Usp alone; EcR and Usp (EcR/Usp); and EcR and Retinoid X Receptor (EcR/RXR). Assays were also performed on a mixture of extracts derived from yeast expressing EcR and Usp alone (EcR+Usp).

FIG. 3A is a graphic representation of saturation analysis of ³H-Ponasterone A binding to an extract derived from a yeast strain co-expressing EcR and Usp. T, total binding; S, specific binding; NS, non-specific binding.

FIG. 3B is a Scatchard plot of ³H-Ponasterone A binding to an extract derived from a yeast strain co-expressing EcR and Usp, showing a K_{d} of 1.8 nM.

FIG. 4 is a graphic illustration of ³H-Ponasterone A binding to an extract derived from a yeast strain co-expressing EcR and Usp, in the presence of increasing amounts of unlabelled Ponasterone A (Pon.A); Muristerone A (Mur.A), ecdysone, and compound 210,230 (RH5949).

FIG. 5 is a graphic illustration of the effect of different solvents on ³H-Ponasterone A binding to an extract derived from a yeast strain co-expressing EcR and Usp.

FIG. 6 is a table showing the effect of different fermentation media on ³H-Ponasterone A binding to an extract derived from a yeast strain co-expressing EcR and Usp.

FIG. 7 is a table showing the effect of different test compounds on ³H-Ponasterone A binding to an extract derived from a yeast strain co-expressing EcR and Usp.

### DETAILED DESCRIPTION OF THE INVENTION

The present invention includes methods and compositions for identifying compounds that bind to and/or activate the ecdysone receptor (EcR). Preferably, compounds identified by the methods of the invention interact with EcR in a manner that mimics the action of ecdysone and other authentic EcR ligands in vivo. As used herein, “authentic EcR ligands”, including agonists, are compounds that exhibit specific, high-affinity binding to EcR and/or transcriptionally activate ecdysone-regulated genes in an insect; authentic EcR ligands are not limited to molecules having a classic steroid backbone structure. Without wishing to be bound by theory, it is believed that compounds identified by the methods of the present invention will be useful as Insect Growth Regulators (IGRs) and, accordingly, will exhibit insecticide activity.

The present inventors have found that a cytosolic extract prepared from a yeast cell co-expressing Drosophila melanogaster-derived EcR and ultraspiracle protein (Usp) exhibits high-affinity specific binding of ecdysone or EcR ligands. This property reflects the formation in vivo of functional complexes between EcR and Usp. According to the invention, yeast are transformed with expression plasmids encoding EcR and UsP, and the transformed yeast are incubated under conditions that result in high-level expression of both EcR and Usp polypeptides. The cells are then harvested and a cytosolic extract is prepared. Finally, a competitive binding assay is performed in which the ability of test compounds to compete with the binding to the extract of an EcR ligand is measured. New EcR ligands are identified as those compounds capable of competing with a known EcR ligand for binding to the yeast extract.

EcR is a polypeptide of 878 amino acids having an apparent domain structure typical of the nuclear steroid receptor family, including an A/B (transactivation) domain, a C (DNA binding/dimerization/transactivation) domain, a D (nuclear localization) domain, an E (dimerization) domain, and an F domain of unknown function (Koelle et al., Cell 67:59, 1991). In practicing the present invention, any homologue or derivative of EcR may be used that is capable of (i) forming a functional complex with an EcR binding partner and (ii) binding EcR ligands. As used herein, a functional EcR-EcR binding partner complex is one that exhibits specific high-affinity binding of ecdysone or other EcR ligands. Useful EcR derivatives may include polypeptides in which one or more amino acids have been added or deleted relative to the wild-type sequence, or in which one or more amino acids have been replaced with different amino acids that do not prevent the formation of a functional EcR-EcR binding partner complex or otherwise interfere with EcR ligand binding. The ability of an EcR homologue or derivative to form a functional EcR-EcR binding partner complex can be ascertained using the methods of the present invention.

EcR binding partners for use in the present invention include any polypeptides capable of forming a heterodimer with EcR (or with an EcR homologue or derivative), wherein EcR present in the heterodimer is capable of specific high-affinity binding of ecdysone or other authentic EcR ligands. Non-limiting examples of EcR binding partners include Usp, RXRα, and RXR homologues. In a preferred embodiment, the EcR binding partner comprises Usp or homologues or derivatives thereof. Usp is a polypeptide of 508 amino acids having a molecular mass of 55,252 daltons (Henrich et al., Nuc.Acids.Res. 18:4143, 1990; Shea et al., Genes Dev. 4:1128, 1990; Yao et al., Cell 71:63, 1992; Oro et al., Nature 347:298, 1990). Any Usp derivative may be used in practicing the invention, so long as it retains its ability to form a functional EcR-EcR binding partner complex. Useful derivatives of Usp or other EcR binding partners may include polypeptides in which one or more amino acids have been added or deleted relative to the wild-type sequence, or in which one or more amino acids have been replaced with different amino acids that do not prevent the formation of a functional EcR-EcR binding partner complex or otherwise interfere with EcR ligand binding. The ability of an EcR binding partner derivative to form a functional EcR-EcR binding partner complex can be ascertained using the methods of the present invention.

In practicing the present invention, many conventional techniques in molecular biology, microbiology, recombinant DNA, and protein biochemistry are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Transcription and Translation, 1984 (Hames and Higgins eds.); A Practical Guide to Molecular Cloning; the series, Methods in Enzymology (Academic Press, Inc.); and Protein Purification: Principles and Practice, Second Edition (Springer-Verlag, N.Y.).

In practicing the present invention, any suitable recombinant cloning vectors may be used for introducing into yeast DNA sequences encoding EcR and EcR binding partners. Such vectors will often include one or more replication systems for cloning or expression, one or more markers for selection in the host, e.g. prototrophy or antibiotic resistance, and one or more expression cassettes. The inserted sequences may be synthesized by standard methods or isolated from natural sources. Suitable vectors include without limitation YEp and YIp vectors (Hill et al., Yeast 2:163, 1986). Non-limiting examples of yeast promoters that may be present in these vectors to direct the expression of EcR and EcR binding partners include metallothionein promoter (CUP1), triosephosphate dehydrogenase promoter (TDH3), 3-phosphoglycerate kinase promoter, glyceraldehyde-3-phosphate dehydrogenase (GAPDH) promoter, galactokinase (GALI) promoter, galactoepimerase promoter, and alcohol dehydrogenase (ADH) promoter.

Host yeast cells may be transformed by any suitable method, including without limitation methods that employ calcium phosphate, lithium salts, electroporation, and spheroplast formation (Sherman et al., Methods in Yeast Genetics, Cold Spring Harbor Laboratory, 1982). Suitable host cells include without limitation Saccharomyces cerevisiae and Schizosaccharomyces pombe. Any host cell in which specific high-affinity binding of ecdysone or ecdysone-related ligands can be measured may be used in practicing the invention. Host cells may also be modified or manipulated, using well-known genetic or pharmacological means or combinations thereof, with respect to their ability to produce different types of covalent modification of proteins, such as, e.g., phosphorylation (Bai et al., Vitamins Horm. 51:289, 1995).

In practicing the present invention, yeast cells co-expressing (i) EcR or an EcR derivative and (ii) an EcR binding partner or derivative thereof, are grown under conditions in which both EcR and EcR binding partner are expressed at high levels. The cells are harvested, and an extract is prepared containing EcR and EcR binding partner. The extract serves as the source of EcR and EcR binding partner for use in an EcR ligand binding assay. Methods for preparing yeast extracts are well-known in the art and include without limitation: vortexing intact cells with glass beads, with or without detergent treatment; spheroplast formation followed by mechanical or hypotonic lysis; sonication; pressure bombs; and the like. The only requirement is that the ability of EcR and EcR binding partner to form functional complexes be retained in the extract. Preferably, a protease inhibitor cocktail (containing, e.g., PMSF, leupeptin, chymostatin, and pepstatin) is included in the lysis buffer. It will be understood that the minimum useful concentration of EcR and EcR binding partner in an extract is determined by the particular binding assay used (see below).

In practicing the invention, any assay may be used that measures the ability of test compounds to specifically bind to EcR-EcR binding partner complexes present in the extract. Typically, such assays involve measuring the ability of a test compound to compete with the binding of an authentic EcR ligand. Preferably, the authentic EcR ligand is radiolabelled, and the assay measures the ability of test compounds to reduce the amount of radiolabel associated with EcR-EcR binding partner complexes. Non-limiting examples of useful radiolabelled authentic EcR ligands include ³H-ponasterone A, ¹²⁵I-ponasterone A, ³H-20-hydroxyecdysone, ³H-muristerone A, and ³H-RH5849 (Wing, Science 241:467, 1988). ³H-ponasterone A is preferred because of its high affinity for EcR and its ability to be tritiated to a high specific activity (Yund et al., Proc.Natl.Acad.Sci. USA 17:6039, 1978).

In a typical assay, each 200 μl sample contains: (i) about 150-400 μg protein, preferably 200-300 μg protein, derived from the yeast extract; and (ii) about 1-25 nM, preferably 10 nM, of a radiolabelled EcR ligand (if tritiated, having a specific activity between about 20-500 Ci/mmol, preferably 150-250 Ci/mmol); in a compatible buffer, preferably: 10 mM Tris-HCl, pH 8.0, 0.1 mM EDTA, 2 mM dithiothreitol, 10% glycerol. Non-specific binding is measured in the presence of a 100-fold molar excess of an unlabelled EcR ligand, such as, e.g., ponasterone A or muristerone A. Test samples include test compounds being assayed for their ability to compete with radiolabelled ligand for EcR binding. Negative control samples include those in which an equivalent volume of the test sample solvent alone is added.

After incubation under appropriate conditions of time and temperature for ligand binding to reach equilibrium (such as, for example, 1 hour at room temperature or 12-24 hours at 4° C.), EcR-EcR binding partner complexes are physically separated from unbound radiolabeled ligand, and the amount of radioactivity bound to the complexes is quantified. Any suitable method may be used for the separation, including without limitation hydroxylapatite chromatography, filtration through glass fiber filters, adsorption to dextran-coated charcoal, and other methods well-known in the art. Radioactivity is measured using well-known methods, including liquid scintillation counting, gamma counting (where appropriate), and any suitable method. Specific binding is defined as total radioactivity bound —nonspecific binding (i.e., the binding observed in the presence of excess unlabelled ligand). Compounds identified by these methods as EcR ligands are those that result in reduction of specific binding by at least about 50%, preferably by at least about 60% and most preferably by at least about 75%.

The methods of the present invention are preferably practiced in a high-throughput screening mode, allowing a multiplicity of test compounds to be assayed simultaneously. Such compounds may be found in, for example, natural product libraries, fermentation libraries (encompassing plants and microorganisms), combinatorial libraries, compound files, and synthetic compound libraries. For example, synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Alternatively, libraries of natural compounds in the form of bacterial, fungal, plant and animal extracts are available from, for example, Pan Laboratories (Bothell, Wash.) or MycoSearch (NC), or are readily producible. Additionally, natural and synthetically produced libraries and compounds are readily modified through conventional chemical, physical, and biochemical means (Blondelle et al., TibTech 14:60, 1996). EcR binding assays according to the present invention are advantageous in accomodating many different types of solvents and thus allowing the testing of compounds from many sources.

Insecticide Compositions

EcR ligands identified according to the present invention encompass “insect growth regulators” (IGRs) that selectively interfere with normal insect development without affecting vertebrate animals or plants. Accordingly, it is believed that such compounds are particularly suitable for use as insecticides, since they are expected to be environmentally benign.

The insecticide activity of EcR ligands identified using the methods of the present invention is tested using techniques well-known in the art. For example, formulations of each identified compound (see below) may be sprayed on a plant to which insect larvae are then applied; after an appropriate time, the degree of plant destruction by the larvae is quantified.

For use as insecticides, EcR ligands are formulated in a biologically acceptable carrier. Suitable biologically acceptable carriers include, but are not limited to, phosphate-buffered saline, saline, deionized water, or the like. Preferred biologically acceptable carriers are physiologically or pharmacologically acceptable carriers.

The insecticide compositions include an insecticide effective amount of active agent. Insecticide effect amounts are those quantities of the insecticide agents of the present invention that afford prophylactic protection against insect infestation in plants and animals, and which result in amelioration or cure of an existing insect infestation in plants or animals. This insecticide effective amount will depend upon the target insect, the agent, and the host. The amount can be determined by experimentation known in the art, such as by establishing a matrix of dosages and frequencies and comparing a group of experimental units or subjects to each point in the matrix.

For agricultural use, the insecticide active agents or compositions can be formed into dosage unit forms such as, for example, emulsifiable concentrates (EC), suspension concentrates (SC), and water dispersable granules (WDG). For pharmaceutical use, The insecticide active agents or compositions can be formed into dosage unit forms such as for example, creams, ointments, lotions, powders, liquids, tablets, capsules, sprays, or the like. If the insecticide composition is formulated into a dosage unit form, the dosage unit form may contain an insecticide effective amount of active agent. Alternatively, the dosage unit form may include less than such an amount if multiple dosage unit forms or multiple dosages are to be used to administer a total dosage of the active agent. Dosage unit forms can include, in addition, one or more excipient(s), diluent(s), disintegrant(s), lubricant(s), plasticizer(s), colorant(s), dosage vehicle(s), absorption enhancer(s), stabilizer(s), bactericide(s), or the like.

The insecticide agents and compositions of the present invention are useful for preventing or treating insect infestations in plants and animals. Prevention methods incorporate a prophylactically effective amount of an insecticide agent or composition. A prophylactically effective amount is an amount effective to prevent infestation and will depend upon the insect, the agent, and the host. These amounts can be determined experimentally by methods known in the art and as described above. Treatment methods incorporate a therapeutically effective amount of an insecticide agent or composition. A therapeutically effective amount is an amount sufficient to reduce an insect infestation. This amount also depends upon the target insect, the agent, and the host, and can be determined as explained above.

The prophylactically and/or therapeutically effective amounts can be administered in one administration or over repeated administrations. Therapeutic administration can be followed by prophylactic administration, once the initial insect infestation has been resolved.

The insecticide agents and compositions can be applied to plants topically or non-topically, i.e., systemically. Topical application is preferably by spraying onto the plant. Systemic administration is preferably by foliar application or by application to the soil and subsequent absorption by the roots of the plant.

### DESCRIPTION OF THE PREFERRED EMBODIMENTS

The following examples illustrate the invention without limitation.

### EXAMPLE 1

Construction of a S. cerevisiae Strain Co-Expressing EcR and Usp

A. Construction of Yeast Expression Plasmids:

An oligonucleotide encoding the first 14 amino acids of the EcR followed by EcoNI and BspEI restriction sites was inserted into the AflII and KpnI restriction sites of the yeast expression vector, YEpV5 (Mak et al., Rec.Prog.Horm.Res. 49:347, 1994; Salerno et al., Nuc. Acids Res. 24:566, 1996). This linker allows the expression of an in-frame ubiquitin-ecdysone receptor fusion protein under the control of the yeast promoter, TDH3 (Mak et al. Gene 145:129, 1994). The N-terminal portion of the EcR gene (1.93 kb) was excised from the plasmid pMKl (Koelle et al., Cell 67:59, 1991) by EcoNI and Bgl II digestion. The remaining portion of the receptor coding sequence (700 bp) was amplified by PCR to contain unique restriction sites, BglII and BspEl at the 5′ and 3′ ends respectively. These two receptor fragments were ligated to the EcoNI and BspEl sites of the yeast vector, YEpV5. The resultant EcR expression vector, YEpEcR was used to transform the yeast strain BJ2168, and transformants were selected by tryptophan auxotrophy.

For the expresson of Usp, a cassette consisting of the metallothionein promoter (CUP1)—ubiquitin gene (76 amino acids)—a linker containing Eagl and DraIII restriction sites followed by the CYCI terminator was inserted into the BamHI and SphI sites (multiple cloning sites) of the yeast vector, YEp351 (Salerno et al., Nuc. Acids Res. 24:566, 1996). The entire coding sequence of the USP gene (1-52 kb) was amplified by PCR using the plasmid pCF1 (Christianson et al., Biochem.Biophy.Res.Comm. 193:1318, 1993) as a template with unique EagI and Dra III restriction sites engineered at the 5′ and 3′ ends respectively. This PCR fragment was ligated to the EagI and DraIII restriction sites of YEp351. The resulting Usp expression plasmid, YEpcUSP, was used to transform yeast strains BJ2168 or YEpEcR and the double transformant yeast strains were selected by tryptophan and leucine auxotrophy.

B. Transformation of Yeast Strains:

Yeast transformation was performed using well-known procedures (Sherman et al., Methods in Yeast Genetics: A Laboratory Manual, Cold Spring Harbor Laboratory, 1982). The host yeast strain was S. cerevisiae strain BJ2168, which has the genotype MAT α leu2 trp1 ura3-52 prb1-1122 pep4-3 prc1-407 gal2.

In BJ2168 transformed with EcR-encoding and Usp-encoding plasmids, EcR is expressed constitutively. To induce Usp expression, cultures were incubated in the presence of 100 μM cupric sulfate for 2-3 hours to activate the CUP1 promoter.

For the coexpression of EcR and retinoid X receptor (RXRα), the expression plasmid YEpEcR was used to transform the yeast strain expressing the human RXRα receptor as described (Mak et al. Gene 145:129, 1994; Salerno et al., Nuc. Acids Res. 24:566, 1996).

C. Characterization of EcR and Usp Expression:

Yeast transformants carrying either the EcR, USP or both ECR and USP expression plasmids were grown overnight in synthetic drop-out medium until the cell density reached late log phase (OD=1.0 at 600 nm). Cells were harvested and yeast extracts were prepared according to standard protocols (Mak et al., J.Biol.Chem. 264:21613, 1989). Protein samples were electrophoresed on a 10% SDS-PAGE, electroeluted onto nitrocellulose and probed with monoclonal antibodies specific to EcR (Koelle et al., Cell 67: 59, 1991) or Usp (Christianson et al., Biochem.Biophys.Res. Comm. 193:1318, 1993).

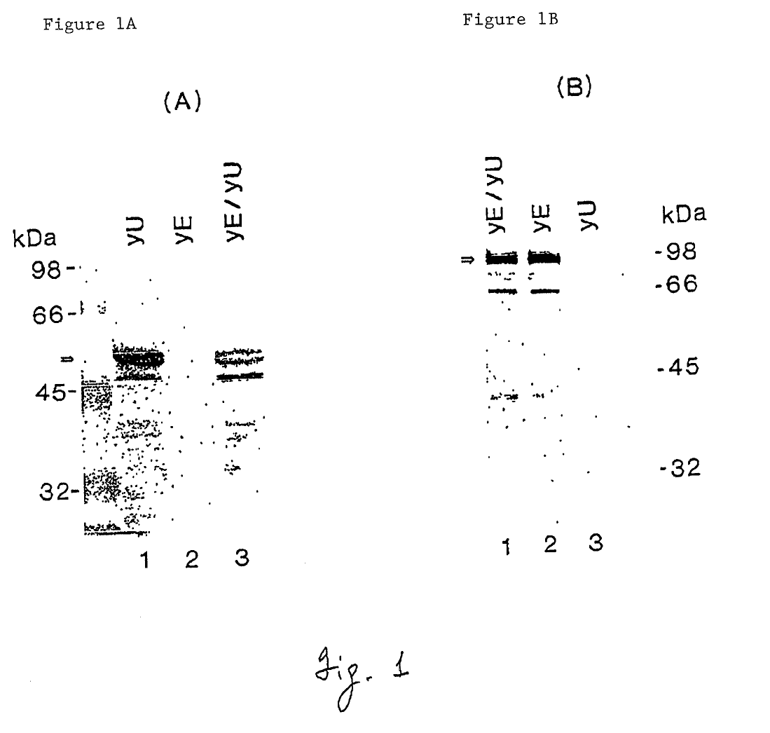

When the blot was probed with the anti-Usp monoclonal antibody AB11, a distinct immunoreactive band with the predicted size of Usp protein (55 kDa) was detected in yeast extracts prepared from yeast strains carrying the Usp expression plasmid alone or both EcR and Usp expression plasmids (FIG. 1A, lanes 1 and 3). This 55 kDa polypeptide was not detected in yeast extract prepared from the yeast strain carrying the EcR expression plasmid (FIG. 1A, lane 2). Minor bands below the 55 kDa polypeptide are most likely degradation products. Concurrently, another blot from the same experiment was probed with the anti-EcR monoclonal antibody AD4.4. An immunoreactive polypeptide corresponding to the full EcR (100 kDa) was detected only in yeast strains carrying the EcR expression plasmids (FIG. 1B, lanes 1 and 2) but not in a yeast strain carrying only the Usp expression plasmid (FIG. 1B, lane 3). These observations indicate that EcR and Usp can be coexpressed in yeast cells. The molecular masses of these recombinant proteins correlate very well with the predicted sizes of the authentic proteins, indicating that the ubiquitin is rapidly cleaved off from the fusion protein by the host enzyme as expected.

### EXAMPLE 2

Binding of EcR Ligands in Yeast Extracts

The following experiments were performed to characterize the binding properties of yeast extracts prepared according to the present invention.

A. Preparation of Extracts:

Overnight cultures of yeast strains expressing EcR, Usp, EcR+Usp, and EcR+RxR were inoculated into 1 liter of selective medium and incubated for 16 h at 30° C. In strains expressing Usp, cupric sulfate was added to a final concentration of 100 μM and the culture incubated an additional 4 h at 30° C. The cells were harvested by centrifugation and washed twice in cold TEDG buffer (10 mM Tris-HCl, pH 8.0, 0.1 mM EDTA, 2 mM dithiothreitol, 10% glycerol). The cell pellet was then resuspended in TEDG buffer containing 0.4M NaCl, and vortexed with glass beads in ten 30-second bursts. The resulting homogenate is centrifuged at 1000×g for 10 min, after which the supernatant is centrifuged at 100,000×g for 30 min. The final supernatant is collected, diluted to 12.5 mg/ml protein with TEDG buffer, and stored in aliquots at −80° C.

B. Binding Assays:

The following components were added to each reaction mixture:

(i) 40 μl yeast extract (12.5 mg/ml protein);

(ii) 20 μl ³H-Ponasterone A (New England Nuclear, 195 Ci/mmol; 20 μl contains 100,000 cpm and a final concentration of 2.5 nM in the reaction mixture);

(iii) 120 μl TEDG buffer containing the following protease inhibitors: PMSF (Sigma Chemical Co.), 0.15 mg/ml; leupeptin (Bachem), 0.04 mg/ml; chymostatin (Bachem), 0.03 mg/ml; and pepstatin (Sigma), 0.01 mg/ml; and

(iv) 20 μl test sample, 25 μM Muristerone A, or control diluent.

The reactions were incubated for 1 h at room temperature or at least 12 h at 4° C. Protein-bound and -free radioactivity were separated by hydroxylapatite chromatography or by filtration, the individual samples suspended in scintillation fluid and the radioactivity determined by scintillation counting.

C. Specificity of Binding:

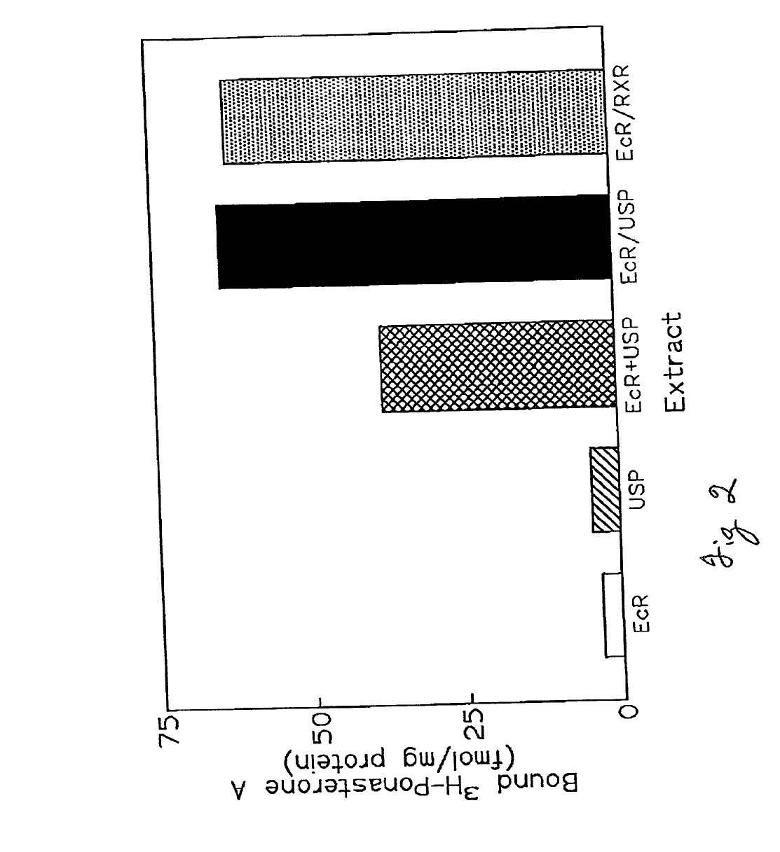

FIG. 2 illustrates the specific binding of ³H-ponasterone A to different yeast extracts. No specific binding could be observed in extracts derived from yeast expressing EcR or Usp alone. By contrast, mixing the two extracts (EcR+Usp) resulted in the appearance of specific binding. Even higher binding activity was observed in extracts prepared from yeast co-expressing EcR and Usp (EcR/Usp) or EcR and Retinoid X Receptor (EcR/RxR).

D. Saturation and Scatchard Analysis:

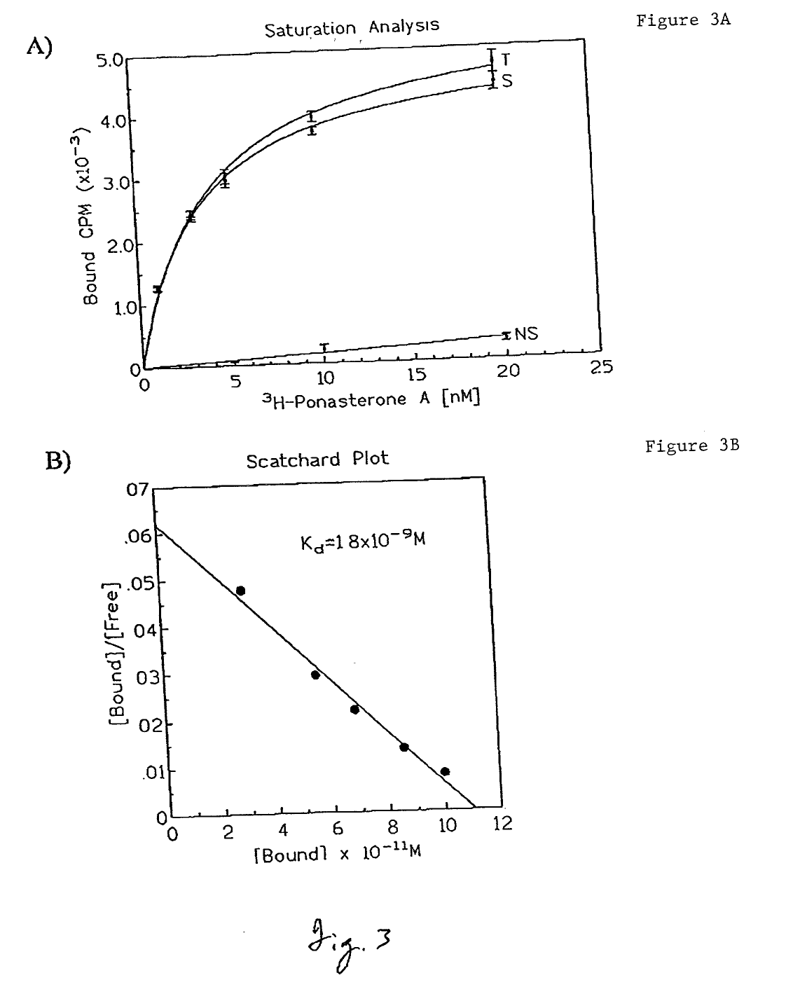

To determine hormone binding affinity and specificity of EcR-Usp complexes, saturation analysis and competition experiments were performed using extracts derived from yeast co-expressing EcR and Usp. Extracts were incubated with ³H-Ponasterone A in the absence or presence of a 100-fold molar excess of unlabelled ponasterone A to determine total (T) and non-specific (NS) binding, respectively. Specific binding (S) is the difference between total and non-specific binding. FIG. 3A shows that specific binding approached saturation at 15-20 nM ³H-ponasterone A. Scatchard analysis (FIG. 3B) revealed a K_{d} of binding of 1.8 nM, which is similar to the value observed in native EcR-expressing insect cells (Cherbas et al., Proc. Natl.Acad. Sci. USA 85:2096, 1988). The number of binding sites in these yeast extracts ranged from 85-120 fmol/mg, which is approximately 10-fold higher than in insect cells.

E. Binding Specificity:

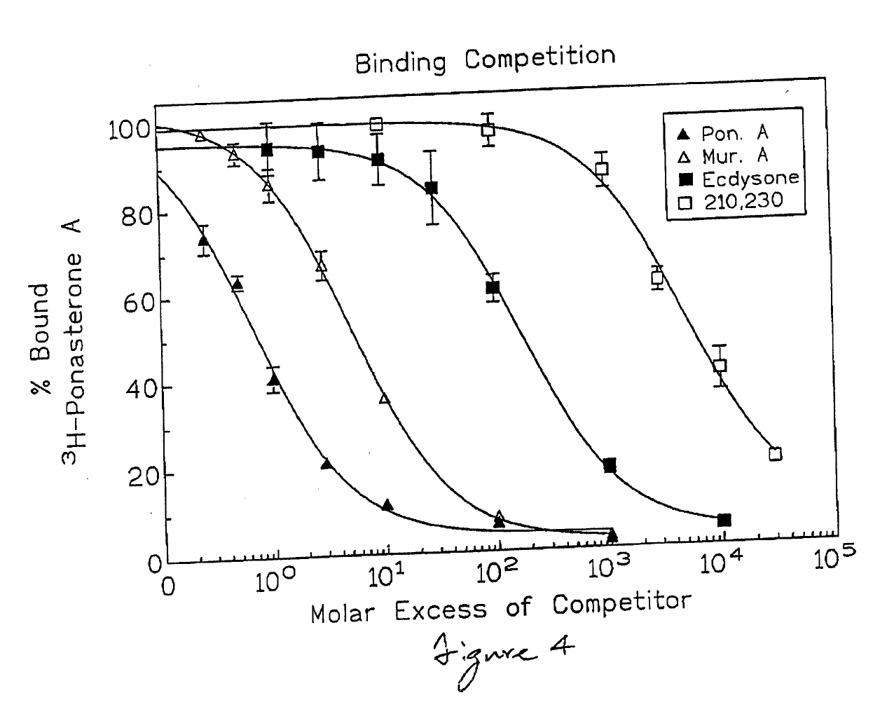

Specificity of hormone binding to the yeast extract was measured by testing the ability of four known (unlabelled) EcR ligands to displace 3H-Ponasterone binding. As shown in FIG. 4, both ponasterone A and muristerone A are excellent competitors, with IC₅₀s of 2.0 nM and 15 nM, respectively. 20-hydroxyecdysone exhibited an IC₅₀ of 0.5 μM, while the non-steroidal EcR agonist RH5849 was much less potent (IC₅₀=12 μM).

F. Compatibility of Solvents:

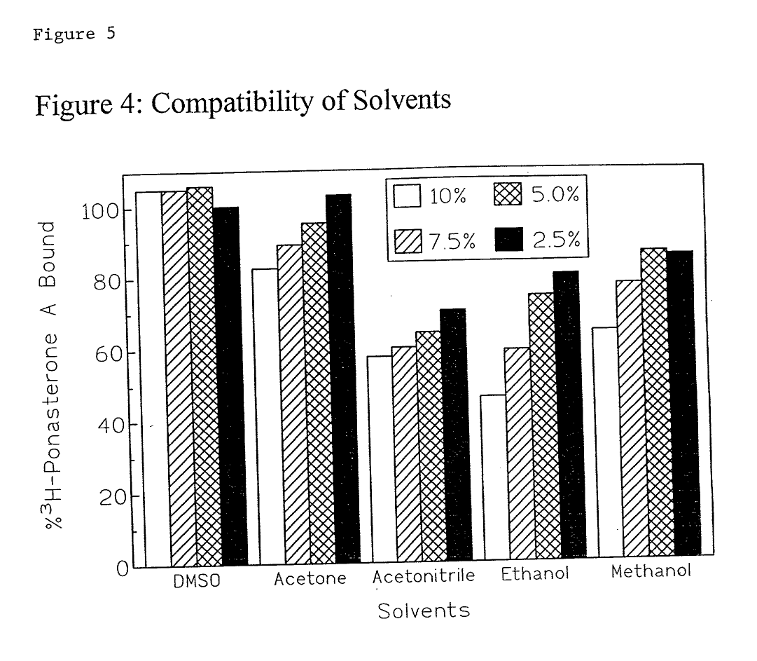

The effect of different solvents on the binding assay was measured by incubating various amounts of each solvent in the binding assay reaction mixture. As shown in FIG. 5, DMSO has no effect on binding up to 10% concentration. Acetone at 2.5% had no effect, but increasing the concentration further caused a dose-dependent decrease in hormone binding. Acetonitrile, ethanol, and methanol had more dramatic effects. Nonetheless, since at least 40-50% binding is retained in the presence of these solvents, they may still be used, so long as appropriate solvent controls are run in parallel.

G. Compatibility of Fermentation Media:

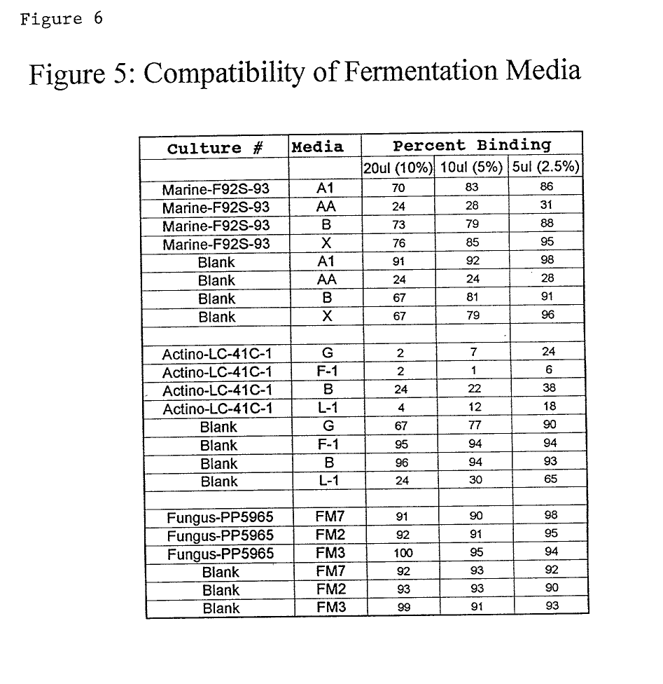

The effect of fermentation broths and media on the binding assay was tested by adding 5, 10, and 20 μl of each broth or medium blank to the reaction mixture (FIG. 6). Of 11 media samples tested, two (AA and L-1) caused a substantial decrease in activity, necessitating the use of appropriate medium controls. The remaining medium samples retaining at least 70% of the binding activity.

The ability of a known EcR ligand, muristerone A, to displace 3H-Ponasterone A was tested in the presence of bacterial and fungal fermentation media; no effect of the medium was observed.

### EXAMPLE 3

Screening Test Compounds for EcR Binding

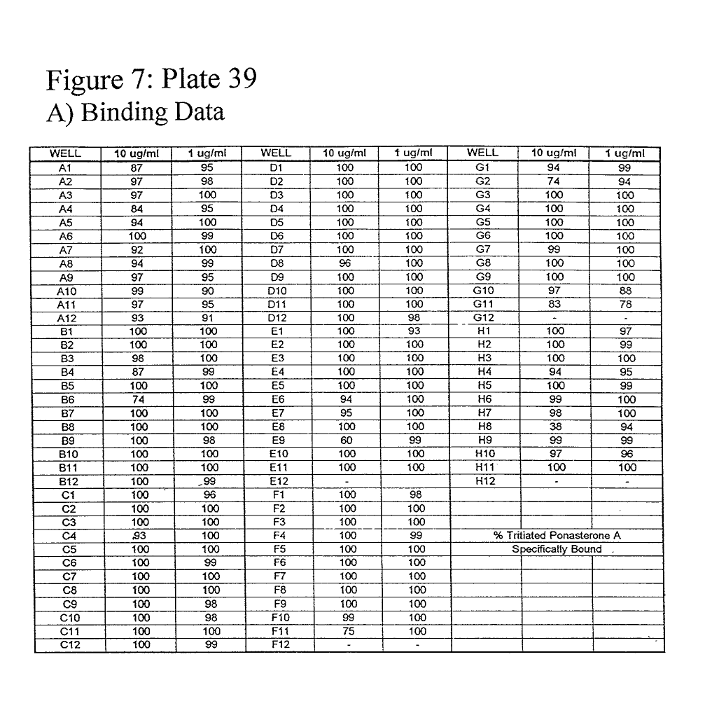

The methods described in Example 2 above were used to test a collection of natural products for EcR binding activity (FIG. 7). The products were tested at 1 and 10 μg/ml. Of 92 compounds tested, one (present in well H8) displaced 62% of ³H-Ponasterone A binding activity at 10 μg/ml.

A chemical library containing 6592 samples was also screened using the methods described above. Each sample was present at 10 μg/ml. Compounds that displaced at least 75% of the radiolabel were scored as positives. These compounds were then used to generate competitive binding curves as described above. Two positive compounds were found to be derivatives of RH5849 (a known EcR ligand). Seven of eight positive compounds were found in dose-response assays to exhibit competitive binding curves, indicating that they were acting on the hormone binding site.

These results indicate that the methods of the present invention can be used in a high-throughput mode to identify EcR ligands.

All patents, patent applications, articles, publications, and test methods mentioned above are hereby incorporated by reference in their entirety.

Many variations of the present invention will suggest themselves to those skilled in the art in light of the above detailed description. Such obvious variations are within the full intended scope of the invention.

In the claims:

1. A method for identifying ecdysone receptor (EcR) ligands, said method comprising:
(i) providing an extract derived from a transformed yeast cell, wherein said extract comprises
(a) EcR or a functional derivative thereof; and
(b) an EcR binding partner or a functional derivative thereof; and
(ii) measuring specific binding to said extract of an authentic EcR ligand, in the absence and presence of a test compound.

2. A method as defined in claim 1 further comprising
(iii) identifying as an EcR ligand a test compound that reduces the specific binding of said authentic EcR ligand to said extract.

3. A method for identifying ecdysone receptor (EcR) ligands, said method comprising:
(i) providing an extract derived from a transformed yeast cell, wherein said extract comprises
(a) EcR or a functional derivative thereof; and
(b) an EcR binding partner or a functional derivative thereof; and
(ii) measuring specific binding to said extract of an authentic EcR ligand, in the absence and presence of a test compound, to identify a test compound that reduces the specific binding of said authentic EcR ligand to said extract.

4. A method as defined in claim 1, wherein said yeast is S. cerevisiae.

5. A method as defined in claim 1, wherein said authentic EcR ligand is selected from the group consisting of 20-hydroxyecdysone, ponasterone, muristerone, and RH5849.

6. A method as defined in claim 1, wherein said measuring is achieved using a competitive binding assay.

7. A method as defined in claim 6, wherein said EcR binding partner is Usp or derivatives thereof.
